(19) United States
(12) Patent Application Publication
(10) Pub. No.: US 2007/0166228 A1
(43) Pub. Date: Jul. 19, 2007

(54) Neutral labeling reactants and conjugates derived thereof

(75) Inventors: Jari Hovinen, Raisio (FI); Jari Peuralahti, Turku (FI); Veli-Matti Mukkala, Kaarina (FI); Kaj Blomberg, Turku (FI)

Correspondence Address: BUCHANAN, INGERSOLL & ROONEY PC, POST OFFICE BOX 1404, ALEXANDRIA, VA, 22313-1404, US

(73) Assignee: WALLAC OY, Turku (FI)

(21) Appl. No.: 11/653,867

(22) Filed: Jan. 17, 2007

Related U.S. Application Data
(60) Provisional application No. 60/759,035, filed on Jan. 17, 2006.

(30) Foreign Application Priority Data
Jan. 17, 2006 (FI) 20065030

(51) Int. Cl. A61K 51/00; A61K 49/10; C07F 13/00; C07F 5/00

(52) U.S. Cl. 424/1.11; 424/9.365; 534/14; 534/16; 530/400

(57) ABSTRACT

This invention concerns novel neutral labeling reactants. The novel reactants are derivatives of diethylenetriaminepentaacetic acid (DTPA) diamides, wherein a suitable group is linked to the molecule allowing introduction of the chelating agent or the neutral chelate to bioactive molecules.

### CROSS-REFERENCE TO RELATED APPLICATIONS

This application claims priority of U.S. Provisional Application No. 60/759,035 filed on Jan. 17, 2006 and claims priority under 35 U.S.C. § 119(e) to such prior application, the disclosure of which is incorporated herein in its entirety by reference. This application also claims priority under 35 U.S.C. § 119 to Finnish Patent Application No. 20065030, filed on Jan. 17, 2006, in the Finnish Intellectual Property Office, the disclosure of which is incorporated herein in its entirety by reference.

### FIELD OF THE DISCLOSURE

This disclosure relates to novel neutral derivatives of diethylenetriaminepentaacetic acid which allow introduction of the said derivatives to bioactive molecules.

### BACKGROUND OF THE DISCLOSURE

The publications and other materials used herein to illuminate the background of the invention, and in particular, cases to provide additional details respecting the practice, are incorporated by reference.

Because of its excellent metal chelating properties diethylenetriaminepentaacetic acid (DTPA) is one of the most widely used organic ligands in magnetic resonance imaging (MRI) and positron emission tomography (PET) [Aime, S., Botta, M., Fasano, M. and Terrano, E. 1998, Chem. Soc. Rev., 27, 19, Caravan, P., Ellison, J. J., McMurry, T. J. and Lauffer, R. B., 1999, Chem. Rev., 99, 2293, Woods, M., Kovacs, Z. and Sherry, A. D., 2002, J. Supramol. Chem., 2, 1]. Indeed, the first FDA approved contrast agent in clinical use is the Gd³⁺ DTPA chelate [Runge, V. M., 2000, J. Magn. Res. Imaging, 12, 205.]. The corresponding ¹¹¹In and ⁶⁸Ga chelates, in turn, are suitable for PET applications [Anderson, C. J. and Welch, M. J., 1999, Chem. Rev. 99, 2219], while Eu³⁺, Tb³⁺, Sm³⁺ and Dy³⁺ chelates can be used in applications based on dissociation enhanced lanthanide fluorescence immunoassay (DELFIA) [PCT WO 03/076939A1]. ^{99m}Tc DTPA in turn, is suitable for single positron emission computed tomography (SPECT) [Lorberboym, M., Lampl, Y. and Sadeh, M., 2003, J. Nucl. Med 44, 1898, Galuska, L., Leovey, A., Szucs-Farkas, Z., Garai, I., Szabo, J., Varga, J. and Nagy, E. V., 2002, Nucl. Med. Commun. 23, 1211]. Bioactive molecules labeled with ¹¹¹In or ^{117m}Sn DTPA may find applications as target-specific radiopharmaceuticals [Volkert, W. A. and Hoffman, T. J., 1999, Chem. Rev. 99, 2269].

In several applications, covalent conjugation of DTPA to bioactive molecules is required. Most commonly this is performed in solution by allowing an amino or mercapto group of a bioactive molecule to react with isothiocyanato, haloacetyl or 3,5-dichloro-2,4,6-triazinyl derivatives of the label molecule. Several bifunctional DTPA derivatives are currently commerically available. Also solid phase methods for the introduction of DTPA to synthetic oligonucleotides [U.S. 6,949,639] and oligopeptides [FI 20055653] have been demonstrated.

The net charge of DTPA chelates is most commonly −2, which may cause problems in several applications. The commonly used MRI contrast agent Gd-DTPA (Magnevist) distributes thorough the extracellular and intravascular fluid spaces, but does not cross an intact blood-brain barrier. Naturally, bioactive molecules labeled with this type of chelates have lower cell permeability than the corresponding intact molecules [Rogers, B. E., Anderson, C. J., Connett, J. M., Guo, L. W., Edwards, W. B., Sherman, E. L., Zinn, K. R., Welch, M. J., 1996, Bioconjugate Chem. 7, 511]. This diminishes the suitability of DTPA chelates to in vivo applications. Furthermore, the negatively charged chelates may bind unselectively to positively charged binding sites of target molecules, such as antibodies, via electrostatic interactions which may result in low recoveries [Rosendale, B. E., Jarrett, D. B., 1985, Clin. Chem., 31, 1965]. Naturally, all these above mentioned problems will be even more serious when the target molecule is labeled with several charged chelates [Peuralahti, J., Suonpää, K., Blomberg, K., Mukkala, V.-M., Hovinen, J. 2004, Bioconjugate Chem. 15, 927].

Several of the above mentioned problems can be avoided by neutralizing the net charge of the chelate by substituting two of the DTPA acetates with carboxamido functions. Indeed, several this type of chelators have been synthesized [Hanaoka, K., Kikuchi, K., Urano, Y., Narazaki, M., Yokawa, T., Sakamoto, S., Yamaguchi, K., Nagano, T. 2002, Chem.Biol. 9, 1027., Feng, J., Sun, G., Pei, F., Liu, M. 2003, Bioorg. Med. Chem. 11, 3359]. The non-ionic derivative, Gd[DTPA-bis(ethylamide)] [Konings, M. S., Dow, W. C., Love, D. B., Raymond, K. N., Quay, S. C., Rocklage, S. M. 1990, Inorg. Chem. 29, 1488], called as gadodiamide (Omniscan) is currently in clinical use. Its osmolality is 40% of that of Gd-DTPA [Lunby, B., Gordon, P., Hugo, F., 1996, Eur. J. Radiol. 23, 190].

It is known that if one of the acetic acid groups of DTPA is used for conjugation, the resulting chelate is less stable than the parent DTPA molecule [Paul-Roth, C. and Raymond, K. N. 1995, lnorg. Chem. 34, 1408, Li, W. P., Ma, D. S., Higginbotham, C., Hoffman, T., Ketring, A. R., Cutler, C. S. and Jurisson, S. S. 2001, Nucl. Med. Biol. 28, 145.]. This may be a serious problem especially in in vivo applications if toxic metal ions have to be used. This has to be taken in account when modifying the metal chelating part of the DTPA molecule.

Several of the above mentioned problems can be avoided by using neutral derivatives of the macrocyclic chelator 1,4,7,10-tetraazacyclododecane-1,4,7,10-tetraacetic acid (DOTA) instead of DTPA in the biomolecule conjugation. However, DOTA is not suitable to all applications. Because of its slow kinetics of chelate formation, the use of DOTA is problematic in applications where short-living radioisotopes are required. In DELFIA assays, in turn, where the chelate has to be rapidly dissociated in acidic conditions, the lanthanide(III) DOTA chelates are too stable.

### SUMMARY OF THE DISCLOSURE

The main object of the present invention is to provide DTPA derivatives, where two of the DTPA acetates are substituted with amides. These chelates do not suffer from the disadvantages of the charged DTPA acetates. Furthermore, the chelating properties of the ligands are practically intact. Accordingly, these new chelates are highly suitable for magnetic resonance imaging (MRI), positron emission tomography (PET), single positron emission computed tomography (SPECT) and dissociation enhanced lanthanide fluorescence immunoassay (DELFIA) as well as target-specific radiopharmaceuticals.

Thus, the present invention concerns a chelate or chelating agent of a formula (I) suitable for labeling of bioactive molecules,

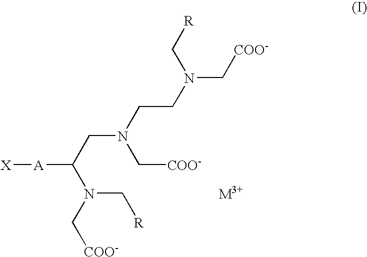

wherein,

—A—is a linker;

R is —CONH₂, —CONHR¹ or —CONR¹R² where R¹ and R², same or different are formed from one to ten moieties, each moiety being selected from the group consisting of phenylene, alkyl containing 1-12 carbon atoms, ethynediyl (—C≡C—), ethylenediyl (—C≡C—); ether (—O—), thioether (—S—), amide (—CO—NH— and —NH—CO— and —CO—NR′ and —NR′—CO—), carbonyl (—CO—), ester (—COO— and —OOC—), disulfide (—SS—), diaza (—N═N—) or a tertiary amine (—NR′—), where R′ represents an alkyl containing less than 5 carbon atoms.

is a reactive group for conjugation of the chelate to a biospecific reactant, wherein said reactive group —X— is selected from amino, aminooxy, haloacetamido, the said halide being preferably bromide or iodide, isothiocyanato, 3,5-dichloro-2,4,6-triazinylamino, maleimido, a thioester or an active ester of a carboxylic acid,
and M is a metal or M is not present.

According to another aspect, the invention concerns a biospecific binding reactant conjugated with the chelate according to this invention.

### BRIEF DESCRIPTION OF THE DRAWINGS

FIG. 1 shows reversed phase HPLC trace of a thyroxine conjugate labeled with a neutral DTPA-Eu(III) chelate (crude reaction mixture). The peak at t_{R} 28.14 min is the desired product as judged on ESI-TOF MS analysis.

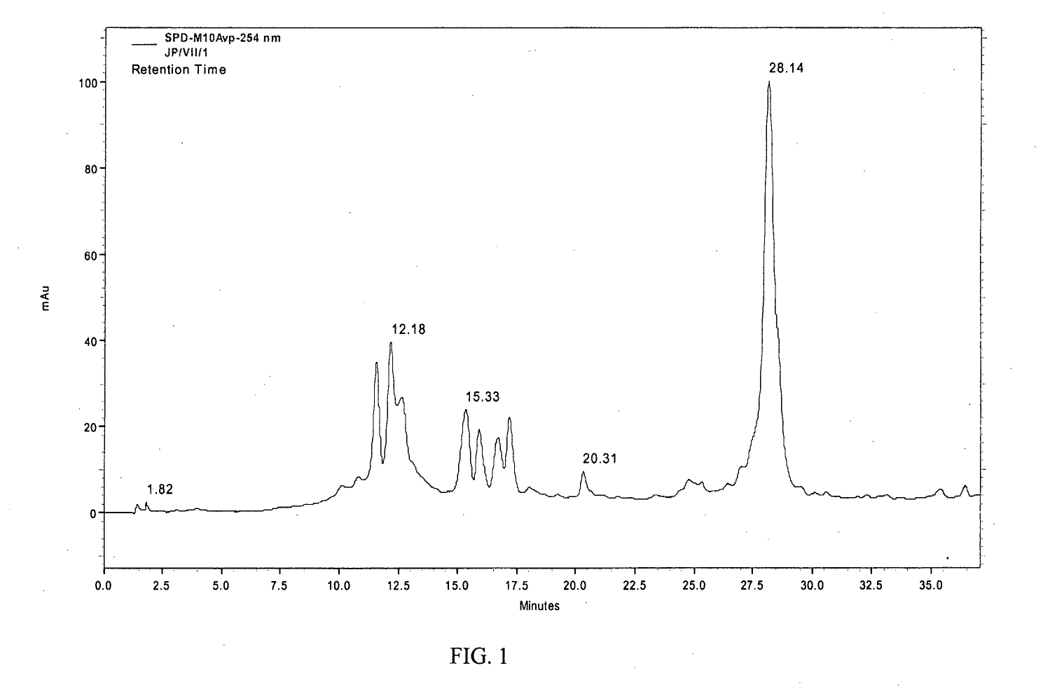

FIG. 2 shows the titration curves of thyroxine (T₄) labeled with various chelates. Open diamonds: 0.35 nM T₄ labeled with the conventional chelate used in AutoDELFIA® Neonatal T₄ kit/0.40 nM Ab; open squares: 0.20 nM T₄-DTPA/0.35 nM Ab; filled diamonds: 0.35 nM 13/30 nM Ab; filled squares: 0.35 nM 13/0.35 nM Ab. The structure of T₄-DTPA is shown in Chart 2.

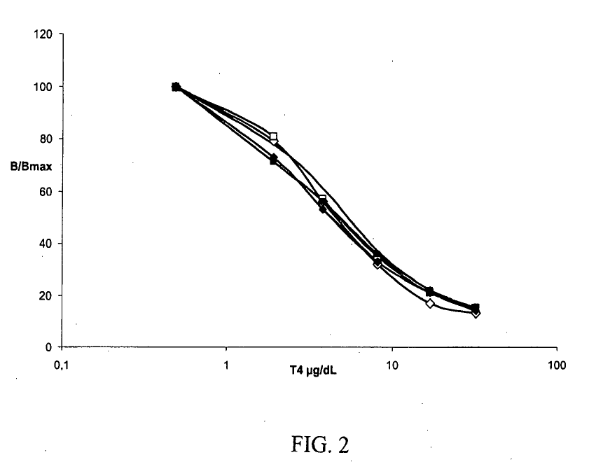

### DETAILED DESCRIPTION

According to a preferable embodiment, the linker —A—is formed from one to ten moieties, each moiety being selected from the group consisting of phenylene, alkyl containing 1-12 carbon atoms, ethynediyl (—C≡C—), ethylenediyl (—C═C—); ether (—O—), thioether (—S—), amide (—CO—NH— and —NH—CO— and —CO—NR′ and —NR′—CO—), carbonyl (—CO—), ester (—COO— and —OOC—), disulfide (—SS—), diaza (—N═N—) or a tertiary amine (—NR′—), where R′ represents an alkyl containing less than 5 carbon atoms.

R is —CONH₂, —CONHR¹ or —CONR¹R² where R¹ and R², same or different are formed from one to ten moieties, each moiety being selected from the group consisting of phenylene, alkyl containing 1-12 carbon atoms, ethynediyl (—C≡C—), ethylenediyl (—C═C—); ether (—O—), thioether (—S—), amide (—CO—NH— and —NH—CO— and —CO—NR′ and —NR′—CO—), carbonyl (—CO—), ester (—COO— and —OOC—), disulfide (—SS—), diaza (—N═N—) or a tertiary amine (—NR′—), where R′ represents an alkyl containing less than 5 carbon atoms.

Where X is an active ester of a carboxylic acid, said ester is preferably an N-hydroxysuccinimido, p-nitrophenol or pentafluorophenol ester.

According to a preferable embodiment the metal M is a metal suitable for use in bioaffinity assays such as a lanthanide or a metal suitable for use in positron emission tomography (PET), single positron emission tomography (SPECT) or magnetic resonange imaging (MRI).

A preferable metal to be used in MRI is gadolinium. However, also lanthanides, particularly europium (III), but also other lanthanides such as samarium (III) and dysprosium (III) are useful in MRI applications. In PET and SPECT applications a radioactive metal isotope is introduced into the chelating agent just before use. Particularly suitable radioactive isotopes are Ga-66, Ga-67, Ga-68, Cr-51, In-111, Y-90, Ho-166, Sm-153, Lu-177, Er-169, Tb-161, Tc-98m, Dy-165, Ho-166, Ce-134, Nd-140, Eu-157, Er-165, Ho-161, Eu-147, Tm-167 and Co-57.

Suitable metals for use in bioaffinity assays are lanthanides, especially europium (III), samarium (III), terbium (III) or dysprosium (III).

The biospecific binding reactant to be labeled is, for example, an oligopeptide, protein, oligosaccharide, polysaccharide, phospholipide, PNA, LNA, antibody, hapten, drug, receptor binding ligand or. lectine. Most preferably, the biospecific binding reactant is an oligopeptide.

The invention will be illuminated by the following non-restrictive Experimental Section.

### EXPERIMENTAL SECTION

The invention is further elucidated by the following examples. The structures and synthetic routes employed in the experimental part are depicted in Schemes 1-3. Experimental details are given in Examples 1-14. Comparison of the stabilities of one of the neutral DTPA chelates and the parent DTPA acetate in DELFIA Enhancement Solution® and in DELFIA Inducer® is shown in Example 15. Structure of the parent DTPA acetate is shown in Chart 1. Example 16 shows the suitability of thyroxine labeled with neutral DTPA Eu(III) chelate in DELFIA based T4-assay. The properties of the new conjugate are compared with the corresponding DTPA acetate as well as with the conventional chelate used in AutoDELFIA® Neonatal T4 kit Structure of the thyroxine tracer labeled with DTPA acetate is shown in Chart 2.

Procedures

Adsorption column chromatography was performed on columns packed with silica gel 60 (Merck) or neutral aluminum oxide (Aldrich; 150 mesh, Brockmann I). 17-α-hydroxyprogesterone 3-CMO and L-thyroxine were purchased from Steraloids and Sigma, respectively. All dry solvents were from Merck and they were used as received. HPLC purifications were performed using a Shimadzu LC 10 AT instrument equipped with a diode array detector, a fraction collector and a reversed phase column (LiChrocart 125-3 Purospher RP-18e 5 μm). Mobile phase: (Buffer A): 0.02 M triethylammonium acetate (pH 7.0); (Buffer B): A in 50% (v/v) acetonitrile. Gradient: from 0 to 1 min 95% A, from 1 to 21 min from 95% A to 100% B. Flow rate was 0.6 mL min⁻¹. NMR spectra were recorded on a Bruker 250 spectrometer operating at 250.13 MHz for ¹H. The signal of TMS was used as an internal reference. ESI-TOF mass spectra were recorded on an Applied Biosystems Mariner instrument. Time-resolved fluorometer VICTOR²V was a product of PerkinElmer LAS.

### EXAMPLES

### Example 1

### The Synthesis of 3-(4-nitrobenzyl)4-oxo-1,9-diphenyl-2,5,8-triazanona-1,8-diene, 2.

2-(4-nitrobenzyl)-3-oxo-1,4,7-triazaheptane (1) (4.6 g, 18.2 mmol), disclosed in Corson, D. T., Meares, C. F., 2000, Bioconjugate Chem. 11, 292, was dissolved to EtOH (45 mL) and the solution was cooled on an ice bath. Benzaldehyde (3.7 mL, 36.5 mmol) was added dropwise and mixture was stirred at ice bath for an hour. Stirring was continued for an additional hour at RT. Solution was dried over Na₂SO₄ filtered and evaporated to dryness. ESI-TOF MS for C₂₅H₂₅N₄O₃⁺(M+H)+: calcd, 429.19; obsd 429.20.

### Example 2

### The Synthesis of 3-(4-nitrobenzyl)-1,9-diphenyl-2,5,8-triazanonane 3

Compound 2 (1.8 g, 4.2 mmol) was dissolved to dry THF (40 mL) and deaerated with argon. The solution was cooled on an ice-water bath, and BH₃-THF-complex (1M, 40 mL) was added dropwise. The solution was allowed to warm to RT and then refluxed overnight. The solution was cooled on ice-water bath and the excess of borane was destroyed by careful addition of water. When foaming had ceased the solution was evaporated to dryness. The residue was dissolved in 20% aq. HCI and refluxed for 3 h, and then stirred overnight at RT. The solution was evaporated to dryness. The residue was partitioned between conc. aqueous ammonia and dichloromethane. The aqueous phase was extracted twice with dichloromethane. The combined organic layers were dried over Na₂SO₄. Purification was performed on neutral AI₂O₃ (eluent, from 0 to 3% methanol (v/v) in CH₂CI₂). ESI-TOF MS for C₂₅H₃₁N₄O₂⁺: (M+H)+: calcd, 419.24; obsd 419.23.

### Example 3

### The Synthesis of 2,5,8-tris(tert-butoxycarbonylmethyl)-3-(4-nitrobenzyl)-1,9-diphenyl-2,5,8-triazanonane, 4

Compound 3 (2.7 g, 6.45 mmol) was dissolved in dry DMF (15 mL). Bromoacetic acid tert-butyl ester (4.8 mL, 32.3 mmol) and DIPEA (9.01 mL, 51.6 mmol) were added and mixture was stirred overnight at RT. The mixture was filtered and the filtrate was evaporated to dryness. Purification was performed on silica gel (eluent, petroleum ether, bp 40-60° C.: ethyl acetate 10:1, v/v). Yield was 3.9 g (79%). ¹H NMR (CDCI₃): δ8.04 (2H, d, J 8.6); 7.37-7.25 (4H, m); 7.20 (2H, d, J 8.6); 7.14-7.02 (6H, m); 3.76 (1H,d,J 13.4); 3.74 (2H, s); 3.66 (1H, d, J 13.7); 3.35-3.27 (3H, m); 3.20 (2H, s); 3.19 (1H, d, J 16.1); 3.04-2.95 (2H, m); 2.89-2.62 (2H, m); 2.38 (1H, dd, J 8.9 and 12.8); 1.46 (9H, s); 1.44 (18H, s). ESI-TOF MS for C₄₃H₆,N₄0₈+(M+H)⁺: calcd, 761.45; obsd 761.41.

### Example 4

### 2,5,8-tris(tert-butoxycarbonylmethyl)-3-(4-aminobenzyl)-1,9-diphenyl-2,5,8-triazanonane, 5

Compound 4 (3.76 g, 4.94 mmol) was dissolved in anhydrous methanol (75 mL). Pd/C (10%, 0.22 g) and sodium borohydride (0.23 g) were added, and the mixture was stirred for 0.5 h at RT and filtered through Celite. The filtrate was neutralized with 1 M HCI and concentrated in vacuo. The residue was suspended in dichloromethane, washed with sat. NaHCO₃ and dried over Na₂SO₄. Purification was performed on silica gel (eluent petroleum ether, bp 40-60° C.: ethyl acetate: triethylamine, from 10:1:1 to 5:1:1, v/v/v)). ¹H NMR (CDCI₃): δ 7.30-7.15 (1OH, m); 6.90 (2H, d, J 8.3); 6.57 (2H, d, J 8.3); 3.82 (1H, d, J 13.9); 3.72 (1H, d, J 13.9); 3.70 (2H, s); 3.52 (2H, s); 3.36 (1H, d,J 17.1); 3.26 (2H, s); 3.25 (1H, d, J 13.9); 3.16 (2H, s); 2.91-2.83 (2H, m); 2.71-2.60 (6H, m); 2.43 (1H, dd, J 8.8 and 14.9); 1.45 (9H, s); 1.43 (9H, s); 1.41 (9H, s). ESI-TOF MS for C₄₃H₆₃N₄0₆+(M+H)+: calcd, 731.47; obsd 731.42.

### Example 5

### The Synthesis of 2,5,8-tris(tert-butoxycarbonylmethyl)-3-(4-tert-butyloxycarbonylaminobenzyl)-1,9-diphenyl-2,5,8-triazanonane, 6

Di-tert-butyldicarbonate (0.68 g, 3.01 mmol) was dissolved in acetonitrile (25 mL) containing triethylamine (420 μL, 3.01 mmol). Compound 5 (2.00 g, 2.74 mmol; predissolved in 8 mL of acetonitrile) was added drop-wise, and the reaction was allowed to proceed for 2 h at RT. All volatiles were removed in vacuo. Purification was performed on silica gel [eluent petroleum ether, bp 40-60° C.: ethyl acetate: triethylamine, 10:1:1 v/v/v)]. ESI-TOF MS for C₄₈H₇₁N₄O₈⁺(M+H)⁺: calcd, 831.53; obsd 831.46.

### Example 6

### The Synthesis of 1,4,7-tris(tert-butoxycarbonylmethyl)-2-(4-tert-butyloxycarbonylaminobenzyl)-1,4,7-triazaheptane, 7

Compound 6 (2.00 g, 2.41 mmol) was dissolved in anhydrous methanol (40 mL) and deaerated with argon. Pd/C (10%; 150 mg) and ammonium formate (0.76 g, 12.03 mmol) were added, and the mixture was heated at reflux for 15 min, before being filtered through Celite and concentrated. Purification was performed on silica gel [eluent petroleum ether, bp 40-60° C.: ethyl acetate: triethylamine, 5:1:1 (v/v/v)]. ESI-TOF MS for C₃₄H₅₉N₄O₈⁺(M+H)⁺: calcd, 651.43; obsd 651.40.

### Example 7

### The Synthesis of 1,7-bis(aminocarbonylmethyl)-1,4,7-tris(tert-butoxycarbonylmethyl)-2-(4-tert-butyloxycarbonylaminobenzyl)-1,4,7-triazaheptane, 8

Compound 7 (0.50 g, 0.77 mmol) was dissolved in dry acetonitrile (5 mL). lodoacetamide (0.26 g, 1.54 mmol) and potassium carbonate (0.42 g, 3.07 mmol) were added, and the mixture was heated at reflux for 5h, before being filtered and concentrated in vacuo. Purification was performed on silica gel [eluent petroleum ether, bp 40-60° C.: ethyl acetate: triethylamine, 2:5:1 (v/v/lv)]. ESI-TOF MS for C₃₈H₇₁N₄O₁₀⁺(M+H)⁺: calcd, 765.48; obsd 765.45.

### Example 8

### The Synthesis of 2-(4-aminobenzyl)-1,7-bis(aminocarbonylmethyl)-1,4,7-tris(carboxymethyl)-1,4,7-triazaheptane, 9

Compound 8 (0.10 g, 0.13 mmol) was dissolved in TFA (5 mL), stirred for 4 h at RT and concentrated. It was used for the next step without further purification.

### Example 9

### The Synthesis of the europium chelate of 2-(4-aminobenzyl)-1,7-bis(aminocarbonylmethyl)-1 ,4,7-tris(carboxymethyl)-1,4,7-triazaheptane, 10

Compound 9 was dissolved in water, and pH was adjusted to 6 with Na₂CO₃. Europium chloride (1.1 eq) was added, and the mixture was stirred for an hour at RT at pH 6. pH of the solution was rised to 8.5, and the europium hydroxide formed was removed by centrifucation. The product was isolated by precipitation upon addition of acetone. ESI-TOF MS for C₂₁H₂₈EuN₆O₈⁺(M−H)³¹ : calcd, 645.18; obsd, 645.11.

### Example 10

### The Synthesis of the Europium Chelate of 1,7-bis(aminocarbonylmethyl)-1 ,4,7-tris(carboxymethyl)-2-(4-isothiocyanatobenzyl)-1,4,7-triazaheptane, 11

Compound 10 (30 mg, 0.046 mmol; predissolved in 200 μL of water) was added to the mixture of thiophosgene (15 μL, 0.19 mmol), Na-HCO₃ (20 mg) and chloroform (400 pL), and the resulting suspension was stirred vigorously for 1 h at RT. The aqueous layer was separated, and washed with chloroform (2 400 μL). The product was isolated by precipitation from acetone. ESI-TOF MS for C₂₂H₂₆EuN₆O₈S³¹ (M−-H)⁻: calcd, 687.07; obsd, 687.01.

### Example 11

### The Synthesis of (5-aminopentylcarboxamido)-L-thyroxine, 12

L-thyroxine (40 mg, 0.05 mmol) was dissolved in the mixture of DMF (2.4 mL) and TEA (320 μL). Fmoc-aminohexanoic acid N-hydroxysuccinate (30 mg, 0.07 mmol) was added, and the mixture was stirred at RT for 1 h in dark. Piperidine (few drops) was added, and the reaction was allowed to proceed for 1 h, before being concetrated in vacuo. The residue was suspended in methanol. The precipitation was isolated by centrifugation and washed twice with methanol. ESI-TOF MS for C₂H₂₃I₄N₂O₅⁺(M+H)⁺: calcd, 890.78; obsd, 890.73

### Example 12

### Labeling of Thyroxine Derivative 12 with the Isothio-Cyanate 11

Compound 11 (15 mg, 17 pmol) was dissolved in the mixture of pyridine, water, and triethylamine (9:1.5:0.1, v/v/v; 100 μL). Compound 12 (15 mg, predissolved in 50 μL of water) was added, and the mixture was stirred for 1 h at RT and concentrated. The residue was suspended in water and precipitated with acetone to yield the desired conjugate 13. Purification was performed on HPLC. ESI-TOF MS for C₄₃H₄₈Eu I₄N₈O₁₃S³¹ (M−H)³¹ : calcd, 1576.85; obsd, 1576.87.

### Example 13

### Synthesis of the 17-α-hydroxyprogesterone Derivative, 14

17-α-hydroxyprogesterone-3-CMO (0.10 g, 0.25 mmol) was dissolved in dioxane (4 mL). DCC (56 mg, 0.27 mmol) and N-hydroxysuccinimide (32 mg, 0.27 mmol) were added, and the reaction was allowed to proceed for 4 h at RT. DCU formed was removed by filtration, and the filtrate was concentrated in vacuo. The residue was redissolved in dioxane (7 mL). Glutamic acid (36 mg, 0.25 mmol; predissolved in 0.1 M NaHCO₃ (7 mL) was added, and the mixture was stirred for 2 h at RT. The precipitation formed was removed by filtration, and the filtrate was concetrated in vacuo. Purification was performed on a preparative TLC plate (eluent, acetonitrile: water, 2:1, v/v). ESI-TOF MS for C₂₆H₃₅N₂O₈⁻(M−H)³¹ : calcd, 503.24; obsd, 503.28.

### Example 14

### Labeling of the Steroid Derivative, 14 with the Amino Chelate 10

Compound 14 (6.5 mg, 12 μmol; predisolved in dioxane) was dissolved in MES-buffer (pH 5.5, 1.5 mL). Compound 10 (16.5 mg, 26 μmol) predissolved in MES buffer (550 μL) was added followed by EDAC (5.0 mg, 26 μmol). The reaction was allowed to proceed for 4 h at RT. Purification was performed on HPLC. ESI-TOF MS for C₆₈H₉₀Eu₂N₁₄O₂₂ (M-2H)²⁻: calcd, 879.23; obsd, 879.23.

### Example 15

### Stabilities of Amino-Eu-DTPA and the Corresponding Neutral Derivative 10 in DELFIA Enhancement Solution® and in DELFIA Inducer®.

The chelates (ca 1 mg) were dissolved either in Inducer or Enhancement Solution. The dissociation of the europium at 25° C. were followed using a time-resolved fluorometer. The results are shown below

TABLE 1
Stabilities of DTPA acetate and the corresponding neutral
derivative 10 at RT. Approximate times needed for complete dissociation.

| chelate | Inducer / mm | Enhancer / mm |
|---|---|---|
| Amino-Eu-DTPA¹ | <5 | 30 |
| Compound 10 | <5 | 30 |

¹Data from PCT WO 03/076939A1

### Example 16

### Comparison of the Performance of the Tracer 13 and the Corresponding DTPA Derivative to AutoDELFIA Neonatal T₄ (Thyroxine) Kit.

The assay concentrations of the antiserum were optimized for each tracer individually, and the analytical sensitivities of the optimized standard curves were defined. The correlation between the methods were studied with a small sample panel. The on-board stability was tested up to one week in instrument-like conditions. Sensitivity to the interference of EDTA-containing samples was also studied. The results are summarised below.

TABLE 2
Comparison of the performance of the tracer 13 and
the corresponding DTPA derivative to AutoDELFIA ® Neonatal T₄ kit.

| | T₄-DTPA | Compound 13 |
|---|---|---|
| Analytical sensitivity | 0.35 μL/dL | 0.42 μL/dL |
| Correlation to | y = 1.24x-3.22, R = | y = 1.02x-0.37, R = |
| AutoDelfia Neonatal T4 | 0.94, n = 27 | 0.87, n = 27 |
| assay | | |
| Mean Bias | −0.1% | −0.7% |
| On board stability | Better | Better |
| Interference with EDTA | No | No |

The shapes of the calibration curves obtained with optimized amounts of tracer and antiserum were slightly different with the three tracers. All tracers were sensitive enough at clinically important range. Assays with the tested tracers compared well to the AutoDELFIA® Neonatal T₄ assay and no significant level differences were obtained.

It will be appreciated that the methods of the present invention can be incorporated in the form of a variety of embodiments, only a few of which are disclosed herein. It will be apparent for the expert skilled in the field that other embodiments exist and do not depart from the spirit of the invention. Thus, the described embodiments are illustrative and should not be construed as restrictive.

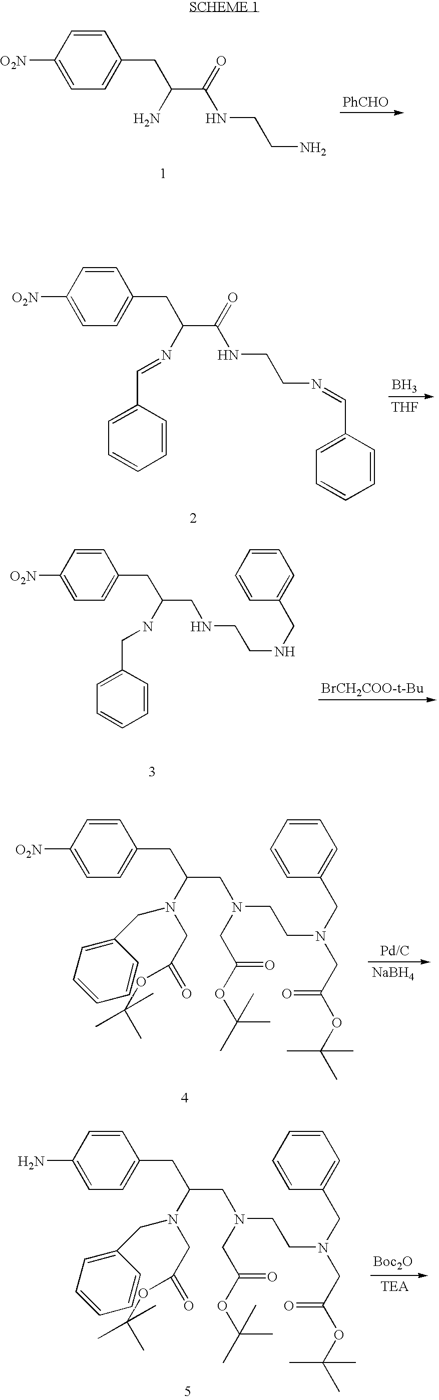

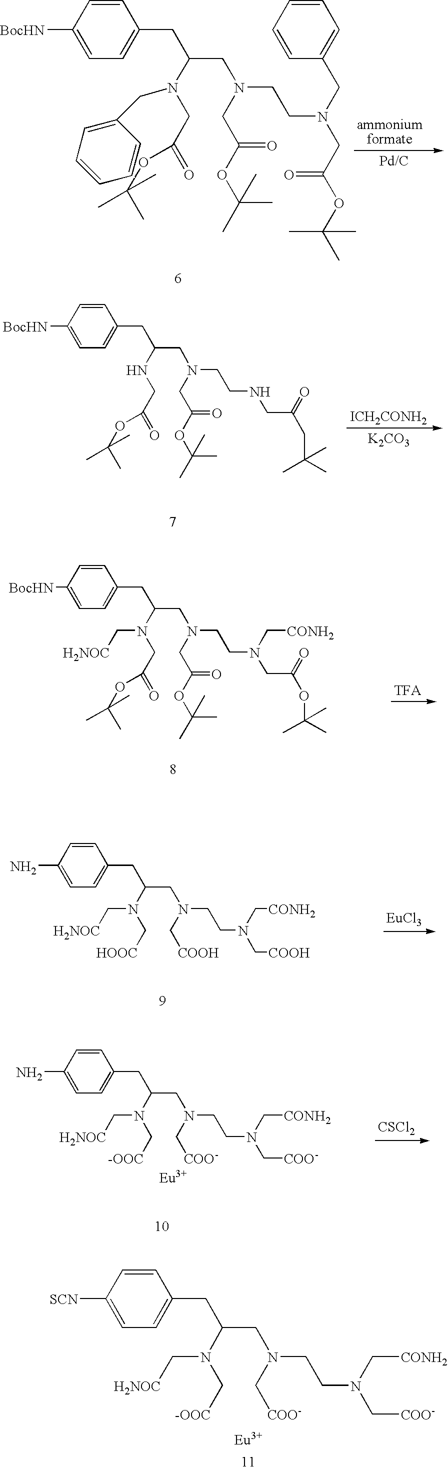

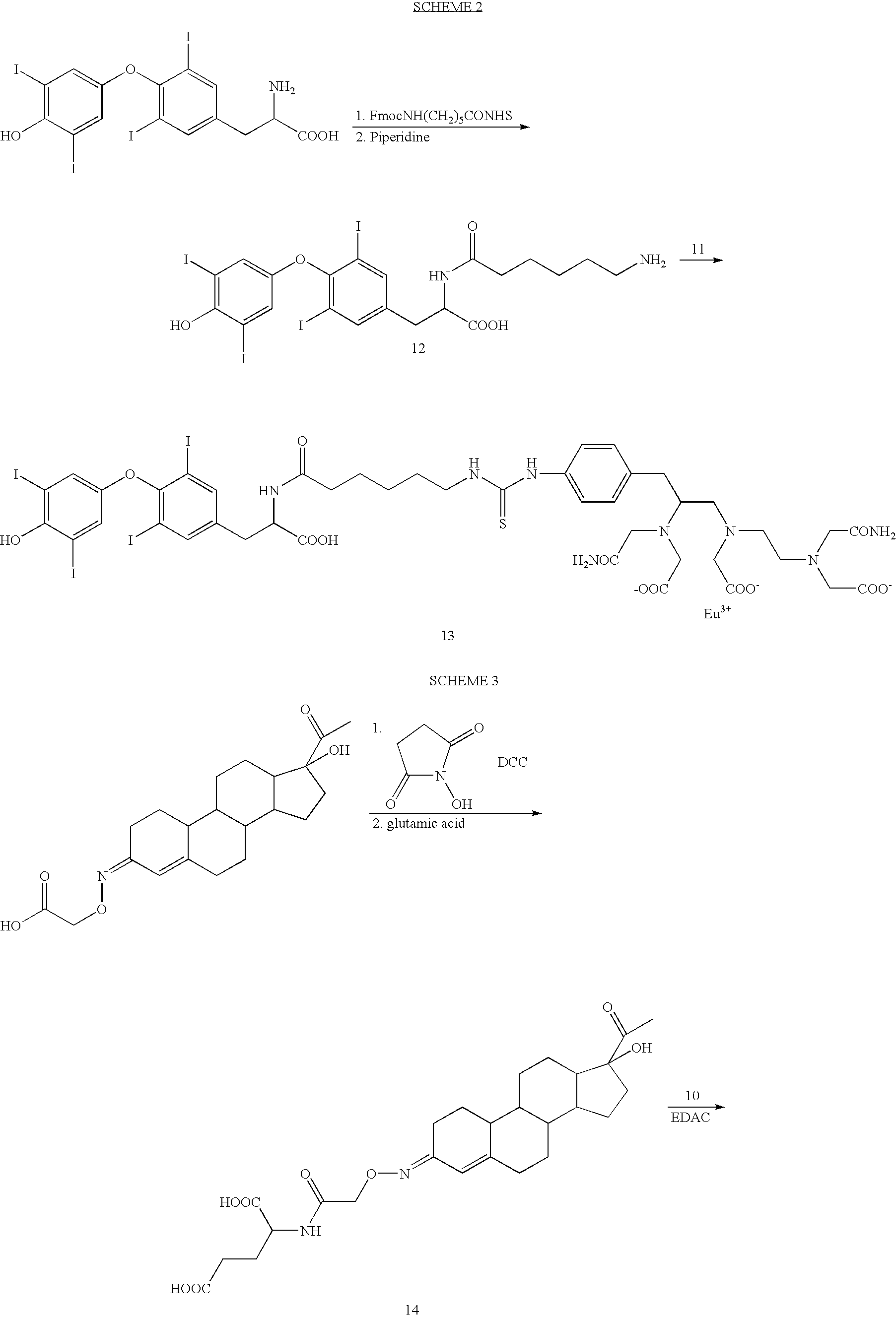

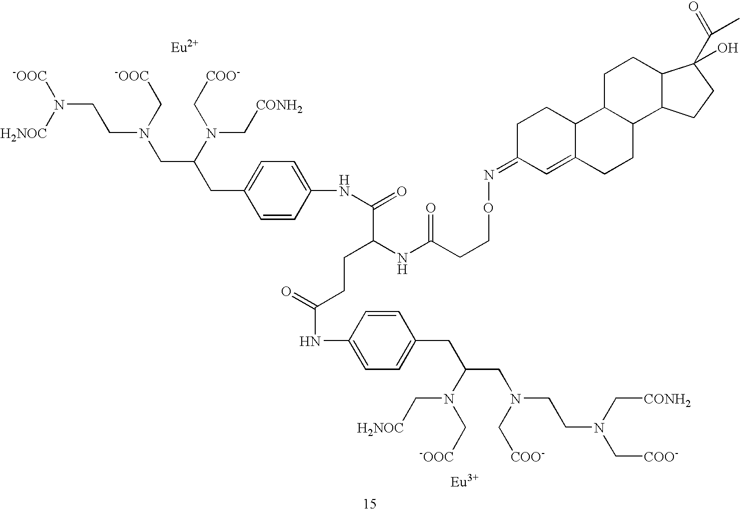

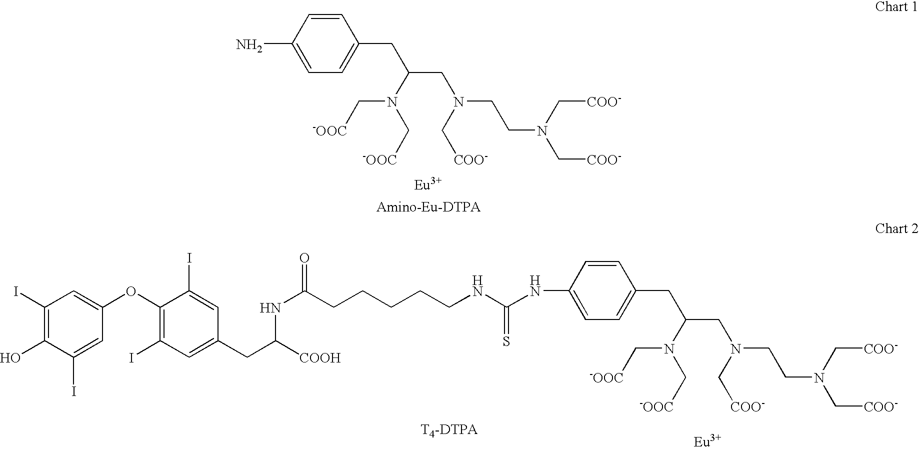

1. A chelate or chelating agent of formula (I) 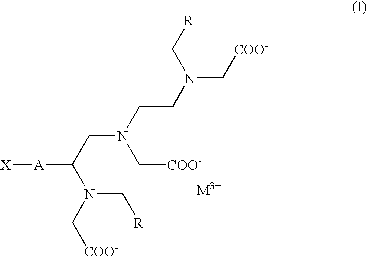
wherein, —A— is a linker;
R is —CONH₂, —CONHR¹ or —CONR¹R² where R¹ and R², same or different are formed from one to ten moieties, each moiety being selected from the group consisting of phenylene, alkyl containing 1-12 carbon atoms, ethynediyl (—C≡C—), ethylenediyl (—C═C—); ether (—O—), thioether (—S—), amide (—CO—NH— and —NH—CO— and —CO—NR′ and —NR′—CO—), carbonyl (—CO—), ester (—COO— and —OOC—), disulfide (—SS—), diaza (—N═N—) or a tertiary amine (—NR′—), where R′ represents an alkyl containing less than 5 carbon atoms;
X is a reactive group for conjugation of the chelate to a biospecific binding reactant, wherein said reactive group X is selected from amino, aminooxy, haloacetamido, isothiocyanato, 3,5-dichloro-2,4,6-triazinylamino, maleimido, a thioester or an active ester of a carboxylic acid,
and M is a metal or M is not present.

2. The chelate or chelating agent according to claim 1 wherein the reactive group X is haloacetamido, where the halide is bromide or iodide,
or wherein X is an active ester of a carboxylic acid and said ester is an N-hydroxysuccinimido, p-nitrophenol or pentafluorophenol ester.

3. The chelate or chelating agent according to claim 1 where the linker A is are formed from one to ten moieties, each moiety being selected from the group consisting of phenylene, alkyl containing 1-12 carbon atoms, ethynediyl (—C≡C—), ethylenediyl (—C═C—); ether (—O—), thioether (—S—), amide (—CO—NH— and —NH—CO— and —CO—NR′ and —NR′—CO—), carbonyl (—CO—), ester (—COO— and —OOC—), disulfide (—SS—), diaza (—N═N—) or a tertiary amine (—NR′—), where R′ represents an alkyl containing less than 5 carbon atoms.

4. The chelate according to claim 1 wherein the metal is a metal suitable for use in bioaffinity assays, especially a lanthanide, or a metal suitable for use in positron emission tomography, single positron emission to mography or magnetic resonance imaging.

5. The chelate according to claim 4 where the metal is gallium-67, gallium-68, technetium-98m, europium, terbium, samarium or dysprosium.

6. The chelate according to claim 1 where the chelate is the europium chelate of 2-(4-aminobenzyl)-1,7-bis(aminocarbonylmethyl)-1,4,7-tris(carboxymethyl)-1,4,7-triazaheptane or the europium chelate of 1,7-bis(aminocarbonylmethyl)-1,4 ,7-tris(carboxymethyl)-2-(4-isothiocyanatobenzyl)-1,4,7-triazaheptane.

7. A conjugate comprising a biospecific binding reactant conjugated with a chelate or chelating agent according to claim 1.

8. The conjugate according to claim 7 wherein the biospecific binding reactant is an oligopeptide, protein, deoxyribonucleic acid, ribonucleic acid, oligosaccharide, polysaccharide, phospholipide, PNA, LNA, antibody, hapten, drug, receptor binding ligand or lectine.
